(19) United States
(12) Patent Application Publication
(10) Pub. No.: US 2004/0142339 A1
(43) Pub. Date: Jul. 22, 2004

(54) Methods for analysis of rna

(76) Inventors: Peter R Cook, Oxford (GB); Hiroshi Kimura, Oxford (GB)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/469,437

(22) PCT Filed: Feb. 27, 2002
(86) PCT No.: PCT/GB02/00868

(30) Foreign Application Priority Data
Feb. 28, 2001 (GB) 0104993.1

(51) Int. Cl. C12Q 1/68; C07H 21/04; C12P 19/34

(52) U.S. Cl. 435/6; 435/91.2; 536/25.3

(57) ABSTRACT

The invention relates to the detection and analysis of RNA transcripts, and in particular to methods of characterising the numbers and types of primary RNA transcripts produced in a given cell or tissue.

### FIELD OF THE INVENTION

The invention relates to the detection and analysis of RNA transcripts and in particular to methods of characterising the numbers and types of primary RNA transcripts produced in a given cell or tissue.

### BACKGROUND OF THE INVENTION

Genetic information flows from a gene, through a primary transcript that is processed into a message, and so into protein. The complete sequence of the human genome is now known, techniques are available to determine the total numbers and types of all mature messages and proteins in cell populations. However, relatively little is known about the numbers and types of primary transcripts. The present invention now provides a method which enables analysis of the missing level in the hierarchy of information transfer, namely the numbers and types of nascent transcripts (i.e. those still associated with engaged polymerases). The method is a general technique for quantitative cataloguing of both genic and non-genic transcripts in one or more cell populations. Useful applications of the technique include monitoring of the changes that occur when cells differentiate, or become malignant, or when they are exposed to exogenous agents (e.g. viruses, chemicals, carcinogens, γ-rays, UV light). Comparisons of different transcript profiles (e.g. of cancer cells and their normal precursors) should allow identification of which transcription units have been repressed or activated (e.g. during tumorigenesis), and in turn this should lead to the development of diagnostic probes, and to the identification of possible targets for therapeutic intervention.

Various high-throughput methods—including those based on SAGE (Velculescu et al., 1995; Kinzler et al., U.S. Pat. No. 5,695,937) and ‘microarrays’ (Brown et al., 1998; Iyer et al., 1999; Lockhart and Winzeler, 2000)—are being used to determine the relative numbers of different messages in a cell. These methods provide catalogues of mature messages, but not of nascent transcripts. This follows for two reasons. First, the RNA selected for analysis contains a poly(A) tail, and so does not contain primary transcripts. This bias results because the poly(A)+ transcripts analysed are necessarily genic transcripts, and because non-genic targets are not usually placed on the microarrays or in SAGE databases. However, recent research indicates that non-genic transcripts form a substantial fraction of primary transcripts, and that they can play critical roles in controlling gene expression (Erdmann et al., 1999). Second, it proves impossible to deduce the numbers of nascent transcripts from the number of mature messages, because the relationship is affected by various (unknown) factors like the rate of transcript processing, RNA export from the nucleus, and message turnover (reviewed by Ross, 1995). Consider just one factor—the stability of the mature message in the cytoplasm. As mammalian messages can vary more than fortyfold in stability, changes in a message profile will inevitably give an imperfect view of any underlying change in transcription profile. As a result, almost nothing is known about the relative frequency of transcription of the different genes in a human cell; we do not even know what is the most active gene transcribed by RNA polymerase II.

No high-throughput techniques capable of giving a snapshot of which genes are being transcribed at any moment are yet available. There is thus a need for a high-throughput technique which enables analysis of the primary transcripts produced in a cell or cell population.

### SUMMARY OF THE INVENTION

In accordance with a first aspect of the invention there is provided a method for analysing RNA transcripts produced in a cell or cell population comprising the steps of:

(i) labelling RNA transcripts transcribed in the cell or cell population with a nucleotide analogue;

(ii) purifying the labelled transcripts;

(iii) synthesising first strand cDNA copies of the labelled transcripts; and

(iv) analysing the resultant cDNA to determine the nature of the RNA transcripts produced in the cell or cell population.

The invention further relates to a method for analysing a population of RNA transcripts which comprises:

(i) ligating the 3′ ends of the RNA transcripts to single-stranded oligonucleotide linkers comprising an enzyme recognition site that allows DNA cleavage at a site spaced a defined distance from the recognition site to form linked RNA;

(ii) converting the linked RNA to double-stranded cDNA using a primer complementary to the oligonucleotide linker for synthesis of the first cDNA strand;

(iii) cleaving the resultant cDNA with an enzyme which recognizes the said enzyme recognition site to generate cDNA tags of a defined length; and

(iv) determining the sequence of multiple cDNA tags and thereby analysing the original population of RNA transcripts.

The invention further provides a nucleic acid composition derived from a cell or cell population comprising at least one ditag, wherein each ditag comprises two covalently joined nucleic acid tags in opposite orientation, wherein each tag corresponds to the extreme 3′ end of a primary RNA transcript expressed in said cell or cell population.

The invention further provides a method of detecting and characterising transcripts associated with a specific transcription unit within a pool of RNA, the method comprising steps of:

(i) ligating single-stranded oligonucleotide linkers of the RNA transcripts to form linked RNA;

(ii) synthesising first-strand cDNA copies of the linked RNA using a first-strand synthesis primer complementary to the oligonucleotide linker;

(iii) amplifying a region of the first-strand cDNA using a first primer corresponding to the oligonucleotide linker and a second primer complementary to a region of the specific transcription unit;

(iv) analysing the resultant amplification products and thereby detecting and characterising the transcripts associated with the specific transcription unit present within the population of RNA transcripts.

### DETAILED DESCRIPTION OF THE INVENTION

In a first aspect the invention relates to the detection and analysis of RNA transcripts produced in a cell or cell population by means of the following steps:

(i) labelling RNA transcripts transcribed in the cell or cell population with a nucleotide analogue;

(ii) purifying the labelled transcripts;

(iii) synthesising first strand cDNA copies of the labelled transcripts; and

(iv) analysing the resultant cDNA to determine the nature of the RNA transcripts produced in the cell or cell population.

The method is particularly suitable for use in analysis of primary RNA transcripts, in which embodiment step (iv) comprises analysing the resultant cDNA to determine the nature of the primary RNA transcripts produced in the cell or cell population. In this embodiment, nascent RNA transcripts transcribed in the cell or cell population are labelled with a nucleotide analogue in step (i). Although a preferred application of the method is in the study of primary transcripts, it will be apparent to the skilled reader that the method may also be used in analysis of transcripts at later stages in the pathway of RNA processing, including mature mRNAs.

It is known to label nascent RNA transcripts within a cell with a nucleotide analogue by allowing controlled extension of RNA transcripts actively engaged to an RNA polymerase in the presence of a labelled nucleotide analogue (Iborra et al., 1996 and 1998; Jackson et al., 1998). Incorporation of a labelled nucleotide into the nascent RNA strand provides a useful means for isolating nascent transcripts away from the remainder of the RNA found within the cell. For example, labelled nascent RNA may be separated from total cellular RNA using an antibody specific for the nucleotide analogue, whilst nascent RNA labelled with a biotinylated nucleotide analogue may be purified using streptavidin-magnetic beads.

The present inventors have unexpectedly determined that RNA transcripts incorporating labelled nucleotide analogues are capable of being faithfully copied by the enzyme reverse transcriptase to form a cDNA. The finding that transcripts incorporating labelled analogue nucleotides can be copied into cDNA has led to the possibility of using the powerful techniques available for high-throughput analysis of cDNA populations in order to analyse populations of nascent RNA transcripts.

The method of the invention is critically dependent on labelling of RNA transcripts. When the method is used to analyse a population of primary RNA transcripts, labelling of nascent RNA molecules allows them to be purified from total cellular RNA. Labelling is accomplished by incorporation of a nucleotide analogue into the nascent transcripts. The nucleotide analogue may be essentially any nucleotide analogue having the following properties:

1) capable of being incorporated into an RNA strand, 2) permits selection of labelled versus non-labelled transcripts, and 3) allows faithful copying of the labelled transcript to form a first-strand cDNA.

Suitable nucleotide analogues include, but are not necessarily limited to, analogues which capable of being recognised by specific antibodies (e.g. Br—UTP), analogues such as biotin-CTP which can be recognised via a high specificity binding reaction (i.e. biotin/avidin or biotin/streptavidin binding) and also analogues which are directly detectable as a result of some property, such as fluorescence, luminescence etc, for example fluorescein-UTP.

Labelling of nascent transcripts may be accomplished using techniques known in the art. Suitable approaches are described, for example, by Iborra et al., 1996 and Jackson et al., 1998. In one approach cells are grown for a short period, typically less than 1.25 min, in media containing the nucleotide analogue, so that the analogue is incorporated into nascent RNA. The resulting labelled RNA can then be purified free of non-labelled (e.g. completed) transcripts, for example using an antibody that reacts specifically with the labelled RNA. A different approach involves permeabilizing cells with saponin, and allowing still-engaged polymerases to extend nascent transcripts in the presence of the labelled nucleotide, before labelled RNA is selected as before. The inventors have observed in control experiments that:

(i) nucleotide analogues are incorporated into RNA (using RNAse and inhibitors of the different RNA polymerases),

(ii) essentially all polymerases active in vivo remain engaged after lysis in saponin and able to ‘run-on’ in the presence of a labelled nucleotide, and

(iii) the different analogues are incorporated into the same nascent transcripts as [³H]cytidine.

Therefore, these methods provide a powerful and simple way of labelling nascent transcripts and then purifying them free of other RNA molecules. In practice, cells are most preferably permeabilized and allowed to extend nascent transcripts by ˜25 nucleotides in the nucleotide analogue (See Jackson et al. (1998) for methods for achieving and monitoring such extensions). This degree of extension is long enough to ensure that most nascent transcripts will be tagged, and that the resulting labelled RNA can be purified easily, but short enough that few transcripts terminate during labelling (many transcripts made by RNA polymerase III are only ˜100 nucleotides), and any unwanted fragmentation of nascent transcripts to give two that would be purified is lessened (any 5′ fragments lacking a labelled nucleotide are not selected). In order to produce labelled transcripts at later stages of the RNA processing pathway, and even labelled mature mRNAs, cells may be grown in the presence of the nucleotide analogue for an extended period of time. A proportion of the labelled transcripts will then be processed into mature mRNA and exported to the cytoplasm.

Labelled RNA transcripts are purified, i.e. selected from the pool of total cellular RNA, and then converted to first strand cDNA. In a preferred embodiment first strand cDNA synthesis may be facilitated by ligating single-stranded oligonucleotide linkers to the 3′ ends of the labelled transcripts. First-strand synthesis may then be initiated using an oligonucleotide primer complementary to the linker. The enzyme T4 RNA ligase may be used to attach a donor 5′ phosphate of a single-stranded DNA linker to the 3′ end of RNA in an efficient reaction (see Uhlenbeck and Gumport, 1982). The primary function of the oligonucleotide linker is to provide an anchor sequence for binding of a first strand cDNA synthesis primer. Therefore, the precise nucleotide sequence of the oligonucleotide linker is generally not material to the invention.

The present inventors have surprisingly shown that RNA strands containing analogue bases, such as Br—U, may be faithfully copied into first-strand cDNA using reverse transcriptase. In fact, copying of transcripts containing nucleotide analogues proceeds equally as efficiently as copying of ordinary unlabelled transcripts. The first-strand cDNAs derived from copying of nucleotide analogue labelled transcripts may also be converted to double-stranded cDNA using reverse transcriptase.

Once the labelled RNA transcripts have been converted to cDNA the resultant cDNA population may be analysed in order to determine the composition of the RNA population from which the cDNA was derived. There are distinct advantages gained from the conversion from RNA to cDNA, (i) cDNA is much more stable than RNA, i.e. less susceptible to degradation; and (ii) there are many high-throughput techniques known in the art for characterisation of cDNAs.

A number of different techniques may be used in order to analyse the cDNA population and in particular to determine which sequences are present in the resulting cDNA population. A non-exhaustive list of suitable techniques is given below. This list is included by way of example only and is not intended to be limiting to the invention:

(i) Direct Sequencing of Inserts

The pool of double-stranded cDNAs derived from the labelled RNA transcripts may be cloned into a suitable vector to form a library of cDNA clones. Most preferably the vector will be one that facilitates direct sequencing of the cDNA inserts. It is then a matter of routine to sequence the inserts of each of the clones in the library. The resulting sequence data may be compared to the available databases in order to determine the numbers and types of cDNA clones present in the library. When the starting material is labelled nascent RNA transcripts, the fragments of sequence derived from each of the clones are called ‘nascent transcript tags’ or NTTs, as opposed to expressed sequence tags or ESTs which are derived from poly (A)+ RNA. cDNA libraries derived from nascent RNA are more complex than conventional libraries made from poly(A)+ RNA, which yield ESTs. They contain cDNAs from all transcription units, and each transcription unit will have representatives with 3′ ends differing in length by one nucleotide, as engaged polymerases will be found at every point along a transcription unit. In contrast, conventional cDNA libraries lack such examples of incomplete transcripts and may contain many copies of the same cDNA with the same 3′ end, and so many copies of the same EST. As a result, one transcription unit in a cDNA library derived from nascent transcripts might be represented by 100 NTTs with different 3′ ends, while a conventional library might contain 100 ESTs with the same 3′ ends. In addition, some 3′ termini will be in untranslated regions (at 5′ and 3′ ends, and in introns); no such termini would be found in conventional cDNA libraries. The analysis of NTTs therefore gives a snapshot of where all engaged RNA polymerases are located on the genome. It provides information on relative activity of all transcription units, and on where polymerases ‘pause’ within them.

Direct sequencing has several advantages: it is straightforward, it uses an established approach, and it yields NTTs long enough to be identified uniquely in the genome. At one level it also provides redundancy—some NTTs will share sequences from the same transcription unit. But at another level, these NTTs derived from the same transcription unit will rarely have the same 3′ ends.

(ii) Using Microarrays

Fluorescent tags may be attached to cDNA copies of the labelled transcripts and the tagged cDNAs hybridized to ‘microarrays’. In order to fully characterise the cDNA population the tagged cDNAs will most preferably be hybridized to arrays covering the complete human genome, including all transcription units with their introns, and 5′ and 3′ regions, as well as non-genic transcription units.

(iii) Using SAGE (Serial Analysis of Gene Expression)

The numbers and abundance of different messages in a cell—or cDNAs generated as above—can be catalogued rapidly using SAGE (Velculescu et al., 1995; Zhang et al., 1997; Kinzler et al., U.S. Pat. No. 5,695,937). SAGE is based on two principles: First, short sequence tags of 9-15 nucleotides are generated that contain sufficient information to identify many transcripts; second, many transcript tags may be concentrated into one molecule that is sequenced using an automated sequencer, so that the identity of multiple tags can be uncovered in one sequencing run. The expression pattern of any population of transcripts can be evaluated quantitatively by determining the abundance of individual tags and identifying the gene corresponding to each tag. SAGE profiles for various cell types are now available, including those of cells from normal human pancreas, colorectal epithelium, and non-small cell lung cancer (eg Hibi et al., 1998; see also http://www.ncbi.nlm.nih.gov/SAGE).

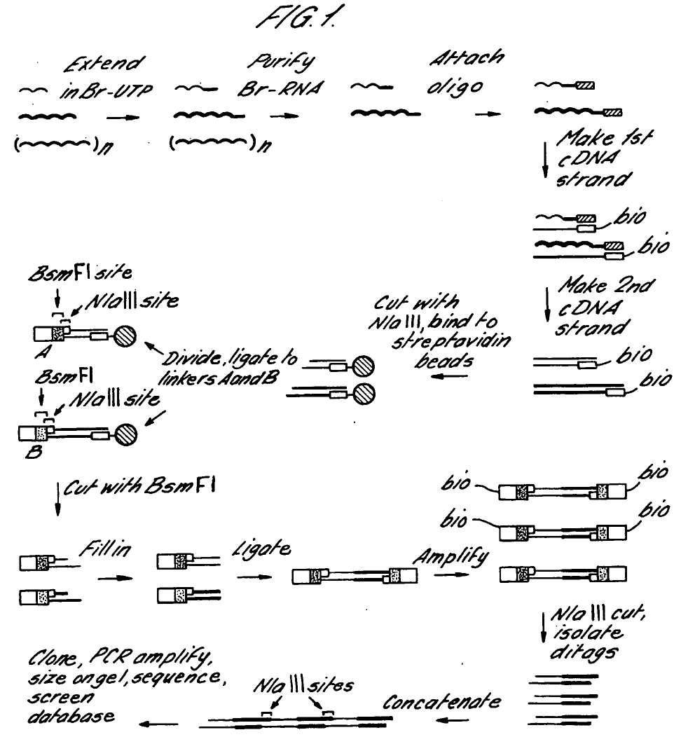

SAGE can be applied directly to the analysis of cDNAs derived from nascent RNA, as illustrated in FIG. 1 by way of example only. The reaction scheme illustrated in FIG. 1 gives a maximum of 15 nucleotides/tag that can be used to screen databases. Conventional SAGE tags derived from poly(A)+ RNA are found next to NlaIII sites at the 3′ end of genes; SAGE tags derived from nascent RNA are found next to NlaIII sites throughout transcription units, thus the SAGE software used to analyse the tag sequences may require adaptation, as will be appreciated by those skilled in the art of bioinformatics. Use of the enzyme BsmFI, as illustrated in FIG. 1, leads to the formation of tags of up to 15 nucleotides in length. Longer tags can be generated using other type IIS restriction enzymes. The production of longer tags provides certain advantages, in particular it is easier to provide unambiguous localization of longer tags within the genome.

(iv) Using Modified SAGE

The present inventors have developed a modified SAGE technique which leads to the production of sequence tags from the extreme 3′ ends of transcripts. This method, described in detail below, is particularly useful in the analysis of nascent RNA transcripts.

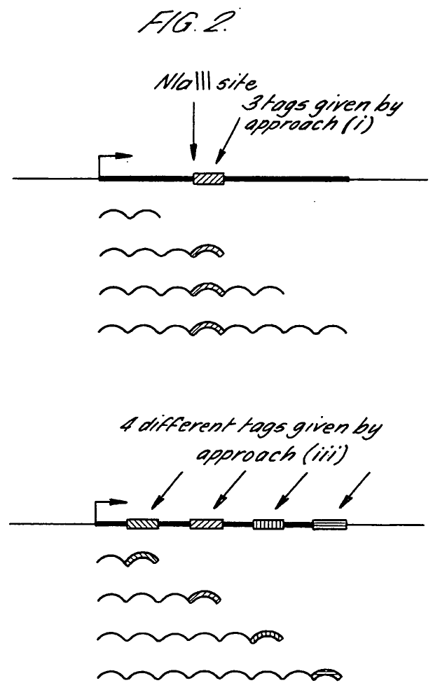

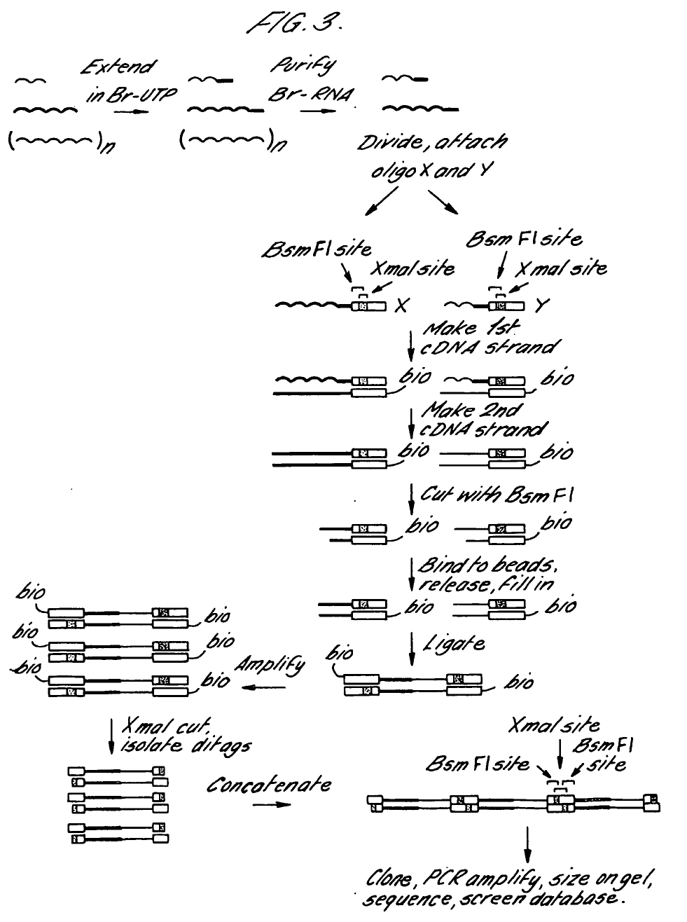

The genome-wide profile obtained using conventional SAGE yields tags next to the most 3′ NlaIII site in transcription units. However, detailed information on the actual 3′ ends of nascent transcripts is lost (FIG. 2, top). The present inventors have developed a method for use in characterising RNA/cDNA populations which is similar to SAGE but which retains information on the 3′ ends of nascent transcripts. Using this method, illustrated schematically by way of example only in FIG. 3, many (sometimes overlapping) NTTs will be derived from each transcription unit so active transcription units can be identified uniquely (FIG. 2, bottom).

Therefore, in accordance with a second aspect of the invention there is provided a method for analysing a population of RNA transcripts which comprises.:

(i) ligating the 3′ ends of the RNA transcripts to single-stranded oligonucleotide linkers comprising an enzyme recognition site that allows DNA cleavage at a site spaced a defined distance from the recognition site to form linked RNA;

(ii) converting the linked RNA to double-stranded cDNA using a primer complementary to the oligonucleotide linker for synthesis of the first cDNA strand;

(iii) cleaving the resultant cDNA with an enzyme which recognizes the said enzyme recognition site to generate cDNA tags of a defined length; and

(iv) determining the sequence of multiple cDNA tags and thereby analysing the original population of RNA transcripts.

The above method is a modified SAGE technique which differs from conventional SAGE techniques known in the prior art in that it leads to the identification of short nucleotide sequence tags from the extreme 3′ ends of RNA transcripts. This is achieved by ligating oligonucleotide linkers containing a recognition site for a tagging enzyme (i.e. an enzyme which cleaves at a defined distance from its recognition site) directly to the 3′ ends of the RNA transcripts. This is quite different to conventional SAGE, in which the linkers containing the recognition sites for the tagging enzyme are joined to the 5′ ends of cDNA fragments generated by cleavage with an anchor enzyme. This fundamental difference will be more fully illustrated with reference to the accompanying Figures.

In a preferred embodiment of the method of the invention, a sample of RNA to be analysed is first divided into two pools of approximately equal size. The first of these RNA pools is ligated to a first oligonucleotide linker, the 5′ ends of the linkers being joined to the 3′ ends of the RNA transcripts, to form a first pool of linked transcripts. The second RNA pool is similarly ligated to a second oligonucleotide linker to form a second pool of linked transcripts. The ligation of a single-stranded DNA oligonucleotide linker to the 3′ end of an RNA transcript may be carried out using the enzyme T4 RNA ligase, according to standard molecular biology protocols.

The first and second oligonucleotide linkers both contain recognition sites for a tagging enzyme, i.e. a restriction enzyme which cuts at a defined distance downstream of its recognition site. The linkers should preferably contain a second restriction enzyme recognition site and most preferably the second restriction site will overlap with the recognition site for the tagging enzyme. The second restriction sites are added to facilitate concatenation of ditags, as described below, and any convenient restriction sites may be used. In a preferred embodiment XmaI sites are used in conjunction with BsmFI tagging enzyme sites. The first and second linkers are preferably DNA oligonucleotides and may be of identical nucleotide sequence or may have different sequences. The precise sequences are not material to the invention, except for the requirement for a tagging enzyme site. If linkers having identical sequences are used it is, of course, possible to carry out the method without first dividing the RNA into two separate pools. If linkers of different sequences are to be used then it is necessary to divide the RNA into two pools prior to the ligation of the linkers. The two pools of linked RNA may then be combined together for the subsequent steps of cDNA synthesis, cleavage with tagging enzyme and ligation to form ditags or may be kept separate up until the ligation step. The skilled person will readily appreciate that such variations may be made without departing from the essential character of the method.

Following ligation of the oligonucleotide linkers to the 3′ ends of the RNA transcripts the linked RNAs are used as templates for synthesis of double-stranded cDNA. Synthesis of the first cDNA strand is primed by first-strand synthesis primers complementary to the oligonucleotide linkers. Advantageously, the first-strand synthesis primers are linked to a capture label which will allow specific capture of the cDNA strands. In a preferred embodiment, the first-strand synthesis primers are conjugated with a biotin capture label which allows capture of the cDNA via biotin/avidin or biotin/streptavidin binding. Other types of capture label may be used with equivalent effect.

The double-stranded cDNAs are cut with the tagging enzyme which cuts a defined distance downstream of its recognition site in the linker sequence added to the 3′ end of the template RNA. As aforesaid, the most preferred tagging enzyme is BsmFI which cuts between ˜10 and ˜14 nucleotides away from its recognition sequence. Treatment with BsmFI releases cDNA fragments, each one containing the linker plus 10-14 nucleotides derived from the extreme 3′ end of one primary transcript. The invention is not limited to the use of BsmFI as the tagging enzyme. The skilled reader will appreciate that other enzymes which share the characteristic of cutting a defined distance away from the recognition site may be used, in particular other type IIs restriction enzymes. As with conventional SAGE, other tagging enzymes may be used which lead to the generation of longer tags. In general, the longer the tags the easier it is to assign them to an unambiguous location within the genome.

Following cleavage with the tagging enzyme, a purification or capture step may be included to separate the tags away from the cDNA fragments cleaved off by the tagging enzyme. In the preferred embodiment wherein a capture label is included in the primer used for first-strand cDNA synthesis the purification step may be easily carried out using a binding agent specific for the capture label. For example, if biotin is used as the capture label then labelled tags may be separated from unlabelled cDNA fragments using avidin or streptavidin coated beads.

Once nucleotide tags have been generated from a defined position in the RNA transcripts and purified/captured, the tags are sequenced to provide an analysis of the original RNA transcripts. As with ‘conventional’ SAGE, it is preferred to concentrate multiple tags into a single molecule to allow high-throughput sequencing of many tags in a single sequencing run. As a first step pairs of cDNA tags are ligated together to form ditags. If the tagging enzyme used to generate the tags was one which generates 3′ or 5′ overhangs, then the overhanging ends must be filled in to generate blunt ends prior to ligation. The ‘filling in’ reaction may be carried out using Klenow polymerase—a standard technique routinely used in molecular biology. Once the ends of the tags have been made blunt, the ligation reaction to form ditags may performed using a DNA ligase, according to standard molecular biology protocols.

Following ligation to form ditags it is preferred to include an amplification step to increase the number of copies of the ditags. The amplification may be performed by conventional PCR using a pair of amplification primers corresponding to regions of the oligonucleotide linkers added to the 3′ ends of the RNA transcripts at the start of the procedure.

In order to further concentrate multiple tags into a single molecule prior to sequencing, individual ditags may be concatenated into chains of ditags. This is achieved by first cleaving the ditags with a restriction enzyme which cleaves at restriction sites within the regions of the ditags derived from the single-stranded oligonucleotide linkers (the second restriction sites described above). If the pool of ditags has been subject to an amplification step then the amplification product is cut with the relevant enzyme. The cleaved ditags may then be concatenated using a conventional DNA ligation reaction.

To facilitate DNA sequencing, the concatamers of ditags are preferably cloned into a standard cloning vector and amplified by conventional PCR. The PCR products may then be sequenced directly and also sized by gel electrophoresis to provide an indication of the number of ditags present in the concatamer.

The modified SAGE technique of the invention is of general applicability and may be used to characterize essentially any pool of RNAs isolated from any cell, cell population or tissue. It is particularly suitable for use in the analysis of nascent RNA transcripts. Therefore, in a preferred embodiment the invention provides a method for analysing the primary transcripts produced in a cell or cell population comprising the steps of:

labelling nascent RNA transcripts transcribed in the cell or cell population with a nucleotide analogue;

purifying the labelled transcripts; and

analysing the labelled transcripts using the modified SAGE method according to the second aspect of the invention.

The steps of labelling nascent RNA transcripts with a nucleotide analogue and purifying the labelled transcripts may be performed as described in connection with the first aspect of the invention.

The invention further provides a nucleic acid composition derived from a cell or cell population comprising at least one ditag, wherein each ditag comprises two covalently joined nucleic acid tags in opposite orientation, wherein each tag corresponds to the extreme 3′ end of a primary RNA transcript expressed in said cell or cell population.

The nucleic acid composition of the invention may be synthesised using the modified SAGE protocol of the invention, up to the step of ligating pairs of tags to form ditags, starting from a pool of nascent RNA isolated from a cell or cell population, for example by selective labelling and purification of nascent RNA as described in connection with the first aspect of the invention. The invention also encompasses nucleic acid compositions comprising concatenated ditags.

In a third aspect, the invention provides a method of detecting and characterising transcripts associated with a specific transcription unit within a pool of RNA, the method comprising steps of:

(i) ligating single-stranded oligonucleotide linkers of the RNA transcripts to form linked RNA;

(ii) synthesising first-strand cDNA copies of the linked RNA using a first-strand synthesis primer complementary to the oligonucleotide linker;

(iii) amplifying a region of the first-strand cDNA using a first primer corresponding to the oligonucleotide linker and a second primer complementary to a region of the specific transcription unit;

(iv) analysing the resultant amplification products and thereby detecting and characterising the transcripts associated with the specific transcription unit present within the population of RNA transcripts.

A second round of PCR amplification using nested or hemi-nested PCR primers may be included between step (iii) and step (iv) is required.

Any of the many techniques known in the art for the detection of PCR amplification products may be used in step (iv). For example, specific sequences may be detected by probing a Southern blot of the PCR products with a probe corresponding to a region of the desired target sequence.

Although the specific method of the invention is of general applicability to the detection and characterisation of specific transcripts from essentially any transcription unit amongst a population of RNA transcripts the method is particularly suitable for the detection and/or characterisation of specific transcripts within a population of nascent RNA. Accordingly, the invention provides a method of detecting and characterising primary transcripts associated with a specific transcription unit within a pool of nascent RNA derived from a given cell or cell type, the method comprising:

labelling nascent RNA transcripts transcribed in the cell or cell population with a nucleotide analogue;

purifying the labelled transcripts; and

detecting and characterising transcripts associated with a specific transcription unit within the labelled transcripts using a method according to the third aspect of the invention.

Since the 3′ ends of nascent transcripts are, by definition, highly variable depending on whereabouts in the gene the RNA polymerase is positioned at a particular point in time known methods such as RT-PCR, which relies on two primers specific for the transcript of interest, and conventional 3′ RACE, which utilises a gene-specific primer and a primer complementary to the poly (A)+ tail, are not suitable for use in characterising nascent RNA transcripts. In contrast, the specific method of the invention can be used for this purpose because of the step of ligating a linker to the 3′ ends of the transcripts.

The invention will be further understood with reference to the following non-limiting experimental examples and the accompanying drawings, in which:

FIG. 1 illustrates a specific example of a method for analysing primary RNA transcripts according to the invention. In this specific example Br—UTP is used as the nucleotide analogue and the double-stranded cDNAs derived from the labelled nascent RNA transcripts are characterised by conventional SAGE.

FIG. 2 illustrates the types of sequence tags which may be generated from four RNA transcripts of varying length using conventional SAGE (approach (i)) or the modified SAGE method of the invention (approach (iii)).

FIG. 3 illustrates a specific example of the modified SAGE method of the invention. In this specific example, Br—UTP is used as the labelled nucleotide analogue, the cDNA synthesis primer is conjugated to a biotin capture label (bio) and BsmFI is used as the tagging enzyme.

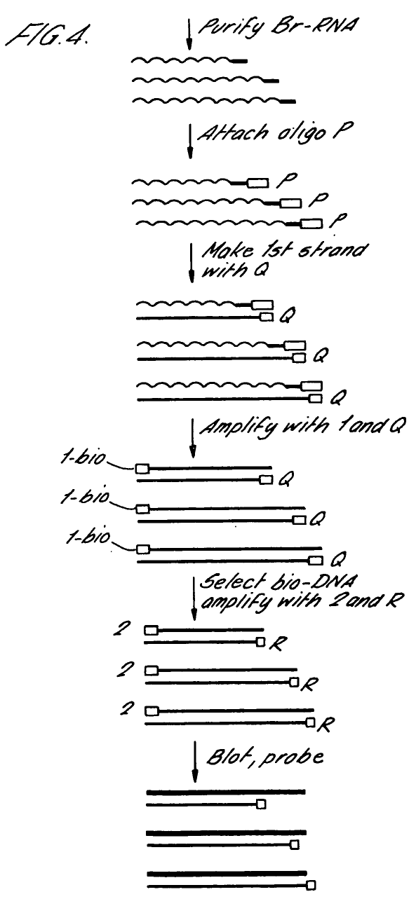

FIG. 4 illustrates a specific example of a method of detecting and characterising transcripts associated with a specific transcription unit according to the invention. This specific example includes a nested PCR step, the final amplification products being detected by Southern blotting.

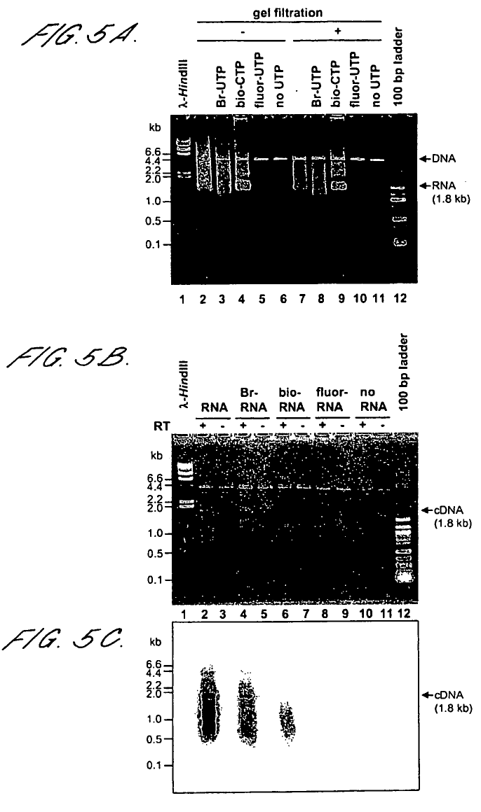

FIG. 5 demonstrates that reverse transcriptase can copy Br-RNA, biotin-RNA and fluorescein-RNA into cDNA.

(A) Illustrates the production of transcripts containing different nucleotide analogs using T7 polymerase, a linear DNA template, and the different nucleotide triphosphates listed:

Lane 1: Marker-8 DNA cut with HindIII.

Lanes 2,7: ATP, CTP, GTP, UTP.

Lane 3,8: ATP, CTP, GTP, Br—UTP (Sigma).

Lane 4,9: ATP, biotin-11-CTP (NEN), GTP, UTP.

Lane 5,10: ATP, CTP, GTP, fluorescein-12-UTP (NEN).

Lane 6,11: ATP, CTP, GTP

Lane 12: Marker-100 bp ladder.

Products of the reactions applied to lanes 7-11 have been subject to gel filtration to remove unincorporated nucleotides. Lanes 2-6 are unfiltered.

The double-stranded DNA template (−4.4 kb) is visible in lanes 2-11. Different amounts of RNA product are seen in lanes 2-5 and 7-9, but little (if any) RNA can be seen in lanes 6, 10, 11. Unincorporated fluorescein-UTP is visible at the bottom of lane 5, but this is removed by gel filtration (lane 10).

(B) Shows an ethidium stained gel illustrating that reverse transcriptase can copy RNA transcripts containing different analogs.

Lanes 1,12: Markers as above.

Lanes 2,3: Normal RNA template (A, lane 7).

Lanes 4,5: Br-RNA template (A, lane 8).

Lanes 6,7: Biotin-RNA template (A, lane 9).

Lanes 8,9: Fluorescein-RNA template (A, lane 10).

Lanes 10,11: No RNA template (A, lane 11).

Denatured template strands (˜4.4 kb) are seen in lanes 2-11. cDNA bands at ˜1.8 kb are visible in lanes 2 and 4 (but not in samples 3 and 5 that lacked reverse transcriptase). Additional cDNA smears of <1.8 kb are visible in lanes 2, 4, 6.

(C) Shows an autoradiograph corresponding to the ethidium stained gel of 5B.

The arrow indicates a cDNA of ˜1.8 kb. All RNA templates give cDNA smears of <1.8 kb in the presence of reverse transcriptase (lanes 2,4,6,8). The bands at ˜4.4 kb (lanes 2,4,6) probably represent short labelled cDNAs hybridized to denatured DNA strands of ˜4.4 kb.

### EXPERIMENTAL EXAMPLES

Molecular biology techniques which may be used in carrying out the invention are described in standard texts such as, for example, F. M. Ausubel et al. (eds.) Current Protocols in Molecular Biology, John Wiley & Sons, Inc. (1994); and Sambrook, J., Fritsch, E. F. and Maniatis, T. (1989). “Molecular cloning: a laboratory manual.’ 2nd Edition. Cold Spring Harbor Laboratory Press, New York.

### Example 1

### To Illustrate Copying of Transcripts Containing Nucleotide Analogues into cDNA by Reverse Transcriptase

The following experiments were performed to see which nucleotide analogs in a transcript could be copied by reverse transcriptase into cDNA (H. Kimura and P. R. Cook, unpublished). In the first, a normal transcript—and one in which every U was substituted with Br—U—were made by allowing T7 RNA polymerase to transcribe a linear template in ATP, CTP, and GTP, plus UTP or Br—UTP. [The linear template (˜4.4 kbp) encoded the luciferase gene (1.8 kbp) under the control of a promoter for T7 RNA polymerase, plus 30 bp poly(dA).] Transcripts and template were now purified free of unincorporated nucleotides. Then, electrophoresis on an agarose gel showed that UTP and Br—UTP both supported the synthesis of ‘run-off’ transcripts of −1.8 kb (FIG. 5A; compare lane 2 with 3, and 7 with 8).

The resulting RNA or Br-RNA with poly(A) at its 3′ end was incubated±reverse transcriptase in the presence of oligo(dT)₁₅, dATP, dGTP, dTTP, plus [³²P]dCTP. Next, samples were denatured, and products separated from unincorporated nucleotides and primers. Copying by reverse transcriptase was monitored by sizing the resulting cDNA strands on an agarose gel. After staining with ethidium, RNA and Br-RNA gave similar distributions of cDNA strands; these distributions extended in size up to ˜1.8 kb, the length of complete copies (FIG. 5B, lanes 2,4) and they were not seen when reverse transcriptase was omitted from the reaction (FIG. 5B, lanes 3,5). This result was confirmed by autoradiography (FIG. 5C, lanes 2-5). As roughly the same amounts of RNA and Br-RNA were added to the reactions (FIG. 5A, lanes 7,8), and as the patterns of the resulting cDNA strands were roughly equivalent, reverse transcriptase must copy Br-RNA into cDNA. However, Br-RNA yielded ˜50% as much cDNA as RNA (FIG. 5C, lanes 2,4). Br-RNA also supported incorporation of [³²P]dCTP (measured by scintillation counting) into acid-insoluble material at a rate (measured over 30 min) of 53% of that given by the natural RNA (not shown).

The same approach was used to generate biotin-RNA and fluorescein-RNA, and then to see if reverse transcriptase could copy these templates into cDNA. T7 RNA polymerase incorporated biotin-CTP into RNA efficiently (FIG. 5A; compare lanes 2 with 4, and 7 with 9). Reverse transcriptase could copy the resulting biotin-RNA into cDNA, however, most of the resulting DNA was shorter than 1.8 kb (FIG. 5B, lane 6; FIG. 5C, lane 6), and the biotin-RNA supported incorporation of [³²P]dCTP into acid-insoluble material at a rate of 10% of that given by the natural RNA (not shown). This shows that reverse transcriptase can copy a template in which every C is replaced by biotin-C, but it does so inefficiently. T7 RNA polymerase incorporated fluorescein-UTP into RNA inefficiently, and yielded little detectable RNA of 1.8 kb (FIG. 5A; compare lanes 2 with 5, and 7 with 10). When the resulting fluorescein-RNA was used as a template for reverse transcriptase, almost no cDNA could be detected by ethidium staining (FIG. 5B, lane 8), but short strands were seen by autoradiography (FIG. 5C, lane 8). The fluorescein-RNA template supported incorporation of [³²P]dCTP into acid-insoluble material at a rate of 0.7% of that given by the natural RNA (not shown). As oligo(dT)₁₅ will only prime cDNA synthesis using a full-length transcript that contains poly(A) at its 3′ end, this incorporation—though slight—suggests that reverse transcriptase can copy fluorescein-RNA into cDNA reasonably efficiently (as so few full-length transcripts are present).

Reverse transcriptase would be expected to copy a Br-RNA (or fluorescein-RNA) template in which only some of the Us had been replaced by Br—U (or fluorescein-U) more efficiently than one in which every U had been replaced. Similarly, it would copy a biotin-RNA template in which only some Cs had been replaced by biotin-C more efficiently than one in which every C had been replaced. Partially-substituted RNA templates are generated when transcription is allowed to proceed in the presence of both UTP and Br—UTP, as is the case when cells are incubated in Br—U.

Methods

(A) Generation of Labelled Transcripts

Transcripts containing different nucleotide analogs were generated using the RiboMax Large Scale RNA Production System T7 (Promega). T7 polymerase was allowed to transcribe 1 μg of a linear template in the presence of the different nucleotide triphosphates indicated. The manufacturer's conditions were used, except that each NTP was present at a concentration of 1 mM. After the reaction, the template and resultant transcripts were purified free of unincorporated nucleotides by gel filtration on Sephacryl S-400HR (Pharmacia) in samples applied to lanes 7-11 of FIG. 5A. Samples (1 and 2 μl for unfiltered and filtered samples, respectively) were applied to 1.2% agarose, subjected to electrophoresis, stained with ethidium, and photographed under UV light (FIG. 5A).

(B) cDNA Synthesis and Autoradiography

2 μl samples from the transcription reactions generated in part (A) were incubated (42BC; 30 min) in 20 μl with 1 μg oligo(dT)₁₅, 1 mM dATP, 1 mM dGTP, 1 mM dTTP, 1 mM [³²P]dCTP (37 TBq/ml), 50 μg/ml actinomycin D, ±30 units reverse transcriptase (RT; Promega) as indicated. Reactions were stopped by incubation (5 min) at 99BC and chilling at 4BC for 5 min. After separating products from unincorporated nucleotides and primers on Sephacryl S-400HR, the products were run on a 1.2% agarose gel, stained with ethidium, and photographed under UV light (FIG. 5B).

Nucleic acids in the agarose gel were blotted on to a positively-charged nylon membrane (Amersham) and radioactively detected using a Phosphorimager (FIG. 5C).

References

Bonner, J., Gottesfeld, J., Garrard, W., Billing, R. and Uphouse, L. (1975). Isolation of template active and inactive regions of chromatin. Methods Enzymol. 40, 97-102.

Brown, P. O., Botstein, D. and Futcher, B. (1998). Comprehensive identification of cell cycle-regulated genes of the yeast Saccharommyces cerevisiae by microarray hybridization. Mol. Biol. Cell 9, 3273-3297.

Chelly, J., Concordet, J. P., Kaplan, J. C. and Kahn, A. (1980). Illegitimate transcription: transcription of any gene in any cell type. Proc. Natl. Acad. Sci. USA 86, 2617-2621.

Dear, P. H. and Cook, P. R. (1993). Happy mapping: linkage mapping using a physical analogue of meiosis. Nucl. Acids Res. 21, 13-20.

Erdmann, V. A., Szymanski, M., Hochberg, A., de Groot, N., Barciszewski, J. (1999). Collection of mRNA-like non-coding RNAs. Nucleic Acids Res. 27, 192-195.

Gribnau, J., Diderich, K., Pruzina, S., Calzolari, R. and Fraser, P. (2000). Intergenic transcription and developmental remodeling of chromatin subdomains in the human b-globin locus. Mol. Cell 5, 377-386.

Hibi, K., Liu, Q., Beaudry, G. A., Madden, S. L., Westra, W. H., Wehage, S. L., Yang, S. C., Heitmiller, R. F., Bertelsen, A. H., Sidransky, D. and Jen, J. (1998). Serial analysis of gene expression in non-small cell lung cancer. Cancer Res. 58, 569-5694.

Huang, R. C., Smith, M. M. and Reeve, A. E. (1978). Studies on gene transcription in vitro by analysis of the primary transcripts. Cold Spring Harb. Symp. Quant. Biol. 42, 589-596.

Iborra, F. J., Pombo, A., Jackson, D. A. and Cook, P. R. (1996). Active RNA polymerases are localized within discrete transcription ‘factories’ in human nuclei. J. Cell Sci. 109, 1427-1436.

Ishii, M., Hashimoto, Si, Tsutsumi, S., Wada, Y., Matsushima., K., Kodama, T. and Aburatani, H. (2000). Direct comparison of GeneChip and SAGE on the quantitative accuracy in transcript profiling analysis. Genomics 68, 136-143.

Iyer, V. R., Eisen, M. B., Ross, D. T., Schuler, G., Moore, T., Lee, J. C. F., Trent, J. M., Staudt, L. M., Hudson, J., Boguski, M. S., Shalon, D., Botstein, D. and Brown, P. O. (1999). The transcriptional program in the response of human fibroblasts to serum. Science 283, 83-87.

Jackson, D. A., Bartlett, J. and Cook, P. R. (1996). Sequences attaching loops of nuclear and mitochondrial DNA to underlying structures in human cells: the role of transcription units. Nucl. Acids Res. 24, 1212-1219.

Jackson, D. A., Iborra, F. J., Manders, E. M. M. and Cook, P. R. (1998). Numbers and organization of RNA polymerases, nascent transcripts and transcription units in HeLa nuclei. Mol. Biol. Cell 9, 1523-1536.

Jackson, D. A., Pombo, A., Iborra, F. J. (2000). The balance sheet for transcription: an analysis of nuclear RNA metabolism in mammalian cells. FASEB J. 14, 242-254.

Kenzelmann, M. and Muhlemann, K. (1999). Substantially enhanced cloning efficiency of SAGE (Serial Analysis of Gene Expression) by adding a heating step to the original protocol. Nucleic Acids Res. 27, 917-918.

Liu, X. and Gorovsky, M. A. (1993). Mapping the 5′ and 3′ ends of Tetrahymena thermophila mRNAs using RNA ligase mediated amplification of cDNA ends (RLM-RACE). Nucl. Acids Res. 21, 4954-4960.

Lockhart, D. J. and Winzeler, E. A. (2000). Genomics, gene expression and DNA arrays. Science 405, 827-836.

Pasquinelli, A. E. et al. (2000). Conservation of the sequence and temporal expression of let-7 heterochronic regulatory RNA. Nature 408, 86-89.

Pfeifer, G. P., Chen, H. H., Komura, J. and Riggs, A. D. (1999). Chromatin structure analysis by ligation-mediated and terminal transferase-mediated polymerase chain reaction. Meth. Enzymol. 304, 548-571.

Powell, J. (1998). Enhanced concatemer cloning: a modification to the SAGE (Serial Analysis of Gene Expression) technique. Nucleic Acids Res. 26, 3445-3446.

Reinhart, B. J., Slack, F. J., Basson, M., Pasquinelli, A. E., Bettinger, J. C., Rougvie, A. E., Horvitz, H. R. and Ruvkun, G. (2000). The 21-nucleotide let-7 RNA regulates developmental timing in Caenorhabditis elegans. Nature 403, 901-906.

Ross, J. (1995). mRNA stability in mammalian cells. Microbiol. Revs. 59, 423-450.

Schaefer, B. C. (1995). Revolutions in rapid amplification of cDNA ends: new strategies for polymerase chain reaction cloning of full-length cDNA ends. Anal. Biochem. 227, 255-273.

Stollberg, J., Urschitz, J., Urban, Z. and Boyd, C. D. (2000). A quantitative evaluation of SAGE. Genome Res. 10, 1241-1248.

Thorey, I. S., Cecera, G., Reynolds, W. and Oshima, R. G. (1993). Alu sequence involvement in transcriptional insulation of the keratin 18 gene in transgenic mice. Mol. Cell. Biol. 13, 6742-6751.

Tracy, R. B. and Lieber, M. R. (2000). Transcription-dependent R-loop formation at mammalian class switch sequences. EMBO J. 19, 1055-1067.

Velculescu, V. E., Zhang, L., Vogelstein, B. and Kinzler, K. W. (1995). Serial analysis of gene expression. Science 270, 484-487.

Uhlenbeck, O. C. and Gumport, R. I. (1982). T4 RNA ligase. In ‘The Enzymes XV, Nucleic Acids, Part B’. Ed Boyer, P. D. Academic Press, NY. pp 31-58.

Willard H. F., Lawrence J., Xing Y., Lafreniere R. G., Rupert J. L., Hendrich B. D. and Brown C. J. (1992). The human XIST gene: analysis of a 17 kb inactive X-specific RNA that contains conserved repeats and is highly localized within the nucleus. Cell 71, 515-529.

Winkles, J. A. (1998). Serum- and polypeptide growth factor-inducible gene expression in mouse fibroblasts. Prog. Nucleic Acid Res. Mol. Biol. 58, 41-78.

Wuarin, J. and Schibler, U. (1994). Physical isolation of nascent RNA chains transcribed by RNA polymerase II: evidence for cotranscriptional splicing. Mol. Cell. Biol. 14, 7219-7225.

Zhang, L., Zhou, W., Velculescu, V. E., Kern, S. E., Hruban, R. H., Hamilton, S. R., Vogelstein, B. and Kinzler, K. W. (1997). Gene expression profiles in normal and cancer cells. Science 276, 1268-1272.

1. A method for analysing RNA transcripts produced in a cell or cell population comprising the steps of:
(i) labelling RNA transcripts transcribed in the cell or cell population with a nucleotide analogue;
(ii) purifying the labelled transcripts;
(iii) synthesising first strand cDNA copies of the labelled transcripts; and
(iv) analysing the resultant cDNA to determine the nature of the RNA transcripts produced in the cell or cell population.

2. A method according to claim 1 wherein step (iv) comprises analysing the resultant cDNA to determine the nature of the primary RNA transcripts produced in the cell or cell population.

3. A method according to claim 1 or claim 2 wherein a single-stranded oligonucleotide linker is ligated to the 3′ ends of the labelled transcripts purified in step (ii) and synthesis of the first-strand cDNAs in step (iii) is carried out using an oligonucleotide primer complementary to the oligonucleotide linker

4. A method according to any one of claims 1 to 3 wherein the nucleotide analogue is recognisable by a specific antibody.

5. A method according to any one of claims 1 to 3 wherein the nucleotide analogue is biotin-CTP or Br—UTP.

6. A method according to any one of claims 1 to 3 wherein the nucleotide analogue includes a fluorescent moiety.

7. A method according to claim 6 wherein the nucleotide analogue is fluorescein-UTP.

8. A method according to any one of claims 1 to 7 wherein the step of analysing the resultant cDNA to determine the nature of the RNA transcripts produced in the cell or cell population comprises:
forming double-stranded cDNA from the first-strand cDNA;
cloning the double-stranded cDNA to form a cDNA library; and
analysing the individual clones present in the cDNA library.

9. A method according to claim 8 wherein the step of analysing the individual clones present in the cDNA library comprises sequencing a portion of the cDNA inserts of clones present in the library.

10. A method according to claim 9 wherein portions of the inserts of substantially all of the clones present in the library are sequenced.

11. A method according to any one of claims 1 to 7 wherein the step of analysing the resultant cDNA to determine the nature of the RNA transcripts produced in the cell or cell population comprises hybridising the cDNA to a microarray.

12. A method according to any one of claims 1 to 7 wherein the step of analysing the resultant cDNA to determine the nature of the RNA transcripts produced in the cell or cell population comprises determining the nucleotide sequence of a defined portion of each of the cDNAs.

13. A method according to claim 12 wherein the nucleotide sequence of a defined portion of each of the cDNAs is determined by performing serial analysis of gene expression.

14. A method according to any one of claims 1 to 13 for use in analysis of primary RNA transcripts produced in a cell or cell population, wherein step (i) comprises labelling nascent RNA transcripts transcribed in the cell or cell population with a nucleotide analogue.

15. A method for analysing a population of RNA transcripts which comprises:
(i) ligating single-stranded oligonucleotide linkers comprising an enzyme recognition site that allows DNA cleavage at a site spaced a defined distance from the recognition site to the 3′ ends of the RNA transcripts to form linked RNA;
(ii) synthesising double-stranded cDNA copies of the linked RNA using a primer complementary to the oligonucleotide linker for synthesis of the first cDNA strand;
(iii) cleaving the cDNA synthesised at step (ii) with an enzyme which recognizes the said enzyme recognition site to generate cDNA tags of a defined length; and
(iv) determining the sequence of multiple cDNA tags and thereby analysing the original population of RNA transcripts.

16. A method according to claim 15 wherein prior to step (i) the population of RNA transcripts is divided into two approximately equal pools of transcripts and thereafter step (i) comprises:
ligating first single-stranded oligonucleotide linkers to the 3′ ends of a first pool of transcripts to form a first linked RNA pool and ligating second single-stranded oligonucleotide linkers to the 3′ ends of the second pool of transcripts to form a second linked RNA pool, wherein the first and second oligonucleotide linkers each comprise an enzyme recognition site that allows DNA cleavage at a site spaced a defined distance from the recognition site;
step (ii) comprises:
synthesising a first double-stranded cDNA pool from the first linked RNA pool using a primer complementary to the first oligonucleotide linker for synthesis of the first cDNA strand and synthesising a second double-stranded cDNA pool from the second linked RNA pool using a primer complementary to the second oligonucleotide linker for synthesis of the first cDNA strand;
and step (iii) comprises:
cleaving the first and second cDNA pools with an enzyme which recognizes the said enzyme recognition site to generate cDNA tags of a defined length.

17. A method according to claim 15 or claim 16 wherein the cDNA tags formed in step (iii) are ligated to form ditags.

18. A method according to claim 17 which further comprises the step of amplifying the ditags prior to sequencing.

19. A method according to claim 17 or claim 18 which further comprises the step of forming concatamers of linked ditags prior to sequencing.

20. A method according to any one of claims 15 to 19 wherein the population of RNA transcripts comprises a pool of nascent RNA.

21. A method for analysing the primary transcripts produced in a cell or cell population comprising the steps of:
labelling nascent RNA transcripts transcribed in the cell or cell population with a nucleotide analogue;
purifying the labelled transcripts; and
analysing the labelled transcripts using a method according to any one of claims 14 to 18.

22. A nucleic acid composition derived from a cell or cell population comprising at least one ditag, wherein each ditag comprises two covalently joined nucleic acid tags in opposite orientation, wherein each tag corresponds to the extreme 3′ end of a primary RNA transcript expressed in said cell or cell population.

23. A method of detecting and characterising transcripts associated with a specific transcription unit within a population of RNA transcripts, the method comprising steps of:
(i) ligating single-stranded oligonucleotide linkers of the RNA transcripts to form linked RNA;
(ii) synthesising first-strand cDNA copies of the linked RNA using a first-strand synthesis primer complementary to the oligonucleotide linker;
(iii) amplifying a region of the first-strand cDNA using a first primer corresponding to the oligonucleotide linker and a second primer complementary to a region of a the specific transcription unit;
(iv) analysing the resultant amplification products and thereby detecting and characterising the transcripts associated with the specific transcription unit present within the population of RNA transcripts.

24. A method according to claim 23 wherein the population of RNA transcripts is a pool of nascent RNA.

25. A method of detecting and characterising primary transcripts associated with a specific transcription unit within a pool of nascent RNA derived from a given cell or cell type, the method comprising:
labelling nascent RNA transcripts transcribed in the cell or cell population with a nucleotide analogue;
purifying the labelled transcripts; and
detecting and characterising transcripts associated with a specific transcription unit within the labelled transcripts using a method according to claim 23.
